(19) United States
(12) Patent Application Publication
(10) Pub. No.: US 2018/0177410 A1
(43) Pub. Date: Jun. 28, 2018

(54) METHOD TO MEASURE NON-INVASIVE BLOOD PRESSURE FROM PATIENT SENSOR SYSTEM

(71) Applicant: General Electric Company, Schenectady, NY (US)

(72) Inventor: Otto Valtteri Pekander, Helsinki (FI)

(73) Assignee: General Electric Company, Schenectady, NY (US)

(21) Appl. No.: 15/387,991

(22) Filed: Dec. 22, 2016

(51) Int. Cl. A61B 5/0205; A61B 5/00; A61B 5/022; A61B 5/0404; A61B 5/1455

(52) U.S. Cl. CPC A61B 5/0205; A61B 5/0004; A61B 5/02225; A61B 2562/0219; A61B 5/0404; A61B 5/14551; A61B 5/6801; A61B 5/02233

(57) ABSTRACT

A system and method for enhancing the determination of the blood pressure of a patient is disclosed. The system includes a wireless blood pressure sensing device that obtains blood pressure data from the patient and wirelessly transmits the data to a host system. The host system receives at least one other physiological parameter from the patient and can retrieve historical information about the patient. Based upon the blood pressure data, physiological parameter and historic data, the host system utilizes a multi-parameter algorithm to calculate a remote blood pressure reading. The blood pressure sensing device calculates a local blood pressure reading based upon the obtained blood pressure data. The host system communicates with the blood pressure sensing device to modify operation of the blood pressure sensing device based upon the at least one physiological parameter other than blood pressure, the historic patient information as well as the calculated remote reading.

### BACKGROUND

The present disclosure relates generally to devices for sensing physiological parameters of a patient and, more specifically, to automated, wireless non-invasive blood pressure (NIBP) monitoring device.

Automated blood pressure monitoring is an accepted and, in many cases, essential aspect of patient treatment. Such monitors are now a conventional part of the patient environment in emergency rooms, intensive and critical care units and in operating theaters.

The oscillometric method of measuring blood pressure involves applying an inflatable cuff around an extremity of a patient's body, such as a patient's upper arm. The cuff is inflated to a pressure above the systolic pressure and then the cuff pressure is reduced either continuously or incrementally in a series of small pressure steps. A pressure sensor contained within the cuff measures the cuff pressure, including the pressure fluctuations resulting form the beat-to-beat pressure changes in the artery under the cuff. The data from the pressure sensor is used to compute the patient's systolic pressure, mean arterial pressure (MAP) and diastolic pressure. Although current NIBP methods operate using algorithms that are proven to be effective in most patients, the standard algorithm can be improved by using other patient parameters, such as arterial wall compliance and other physiological parameters measured from the patient or obtained from a stored patient record.

In the current field of medicine, physicians often desire to monitor multiple physiological characteristics of their patients. Oftentimes, patient monitoring involves the use of several separate monitoring devices simultaneously, such as a pulse oximeter, a NIBP monitor, a heart monitor, a temperature sensor, etc. Several separate patient monitoring devices are often connected to a patient, tethering the patient to multiple bulky bedside devices via physical wiring or cables. Multi-parameter monitors are also available in which different sensor sets may be connected to a single monitor. However, such multi-parameter systems may be even more restrictive than separate monitoring devices because they require all of the sensors attached to a patient to be physically attached to a single monitor, resulting in multiple wires running across the patient's body. Thus, currently available patient monitoring devices often inhibit patient movement, requiring a patient to stay in one location or to transport a large monitor with them when they move from one place to another.

Presently, wireless patient monitoring systems are being developed in which one or more wearable sensing devices are positioned on the patient to monitor one or more physiological parameters of the patient. The sensing devices communicate sensed information to one or more patient monitoring hubs. Typically, the wireless sensing devices are battery powered and rely upon the battery to provide communication to the monitoring hub.

Since wireless patient monitoring systems typically monitor more than one physiological parameter of the patient and relay the sensed information to the patient monitoring hub, this patient information is available for use in enhancing the processing of any one of the wearable sensing devices. Further, this combined information can be used in either real-time processing or in historical patient analysis to enhance the treatment and monitoring of a patient.

### SUMMARY

The present disclosure generally relates to a blood pressure monitoring system. More specifically, the present disclosure relates to a wireless blood pressure sensing device that communicates to a host system to enhance the determination of the blood pressure of the monitored patient.

The blood pressure monitoring system includes a blood pressure sensing device that is configured to be worn by a patient. The blood pressure sensing device includes a blood pressure cuff and a sensor that operate to obtain blood pressure data from the patient. The blood pressure data can include oscillation amplitudes measured by the sensor, which can be a pressure transducer. The wireless blood pressure sensing device includes a transceiver that wireles sly transmits the obtained blood pressure data from the wireless blood pressure sensing device.

A host system is positioned to receive the transmitted blood pressure data from the wireless blood pressure sensing device. The host system includes a host processor that is able to retrieve any one of multiple stored processing algorithms that can be used to calculate a remote blood pressure reading for the patient. The processing algorithms can be multi-parameter algorithms that utilize not only the blood pressure data from the patient but other factors as well.

The host system is configured to receive at least one physiological parameter from a patient sensing device. The physiological parameter is separate and distinct from the blood pressure data. In one illustrative example, the physiological parameter could be an ECG signal. In another illustrative example, the physiological parameter could be a SpO₂ reading. In other examples, the physiological parameter received from the patient could be patient orientation determined from one or more accelerometers, patient temperature, patient heart rate or any other physiological parameter that may enhance the blood pressure determination.

In yet another illustrative example, the host processor of the host system can retrieve historic patient data from a medical record for the patient. The patient data can include past diagnoses for the patient, height, weight, age, family history, past blood pressure readings or any other historic information that may be used to enhance the blood pressure reading made by the host system.

Once the host system receives the multiple parameters in addition to the blood pressure data, the host system utilizes one of the envelope algorithms to calculate a remote blood pressure reading.

In addition to the host system is calculating the remote blood pressure reading, the wireless blood pressure sensing device utilizes the blood pressure data to calculate a local blood pressure reading. The local blood pressure reading is made based solely upon the obtained blood pressure data from the patient. The local blood pressure reading can be compared to the remote blood pressure reading to validate each of the two readings.

The host system can communicate with the wireless blood pressure sensing device to relay modifications to the operation of the blood pressure sensing device. These modifications may include adjusting the target inflation pressure, adjusting the time spent at each discreet pressure step, adjusting the number of oscillation amplitudes recorded at each pressure step along with other operational parameters that may be affected by the other physiological parameters received from the patient or the patient's historical medical record. In this manner, the host system is able to adjust and modify the operation of the wireless blood pressure sensing device to enhance the operation of the wireless blood pressure sensing device.

Various other features, objects and advantages of the invention will be made apparent from the following description taken together with the drawings.

### BRIEF DESCRIPTION OF THE DRAWINGS

The drawings illustrate the best mode presently contemplated of carrying out the disclosure. In the drawings:

FIG. 1 is an schematic illustration of a patient monitoring system including multiple wireless sensing devices and a patient monitoring hub;

FIG. 2 is a schematic illustration of the components contained within the wireless blood pressure sensing device;

FIG. 3 is a sample pressure profile and oscillometric envelope utilized by the blood pressure sensing device; and

FIG. 4 is a flowchart illustrating the operational sequence carried out by the blood pressure sensing device and host system in accordance with one embodiment of the disclosure.

### DETAILED DESCRIPTION

The present inventors have recognized that wireless monitoring systems are desirable for patient comfort, for example to provide more comfort and mobility to the patient being monitored. The patient's movement is not inhibited by wires between sensor devices and/or computing devices that collect and process the physiological data from the patient. Thus, small sensing devices and sensors that can be easily attached to the patient's body are desirable, such as sensing devices that are wearable portable computing devices. In order to do so, the size of the wireless sensing devices must be small.

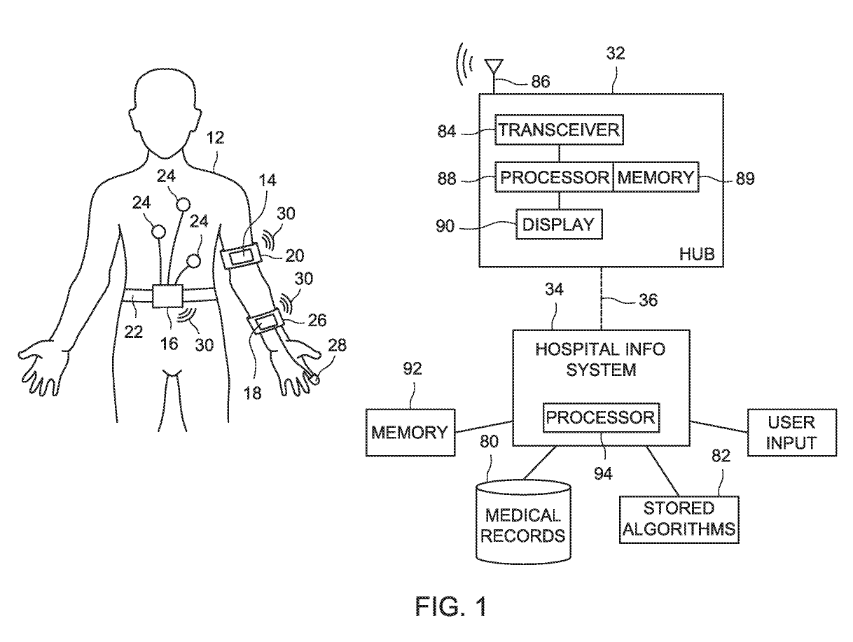

In view of their recognition of problems and challenges in the development of wireless sensing devices, the present inventors developed the disclosed system and method. FIG. 1 illustrates a patient 12 that is being monitored by the system of the present disclosure. In the embodiment shown in FIG. 1, the patient is wearing three separate wireless sensing devices that are each used to monitor different physiological parameters of the patient. In the embodiment shown, the patient is wearing a non-invasive blood pressure sensing device 14, and ECG sensing device 16 and an SpO₂ sensing device 18. The blood pressure sensing device 14 includes a pressure cuff 20 surrounding an arm of the patient 12. The ECG sensing device 16 is attached around the torso of a patient by a strap 22. The ECG sensing device 16 includes a plurality of electrodes 24 that are positioned in desired locations on the patient to receive ECG signals. The SpO₂ sensing device 18 is mounted to a wrist strap 26 and includes a fingertip probe 28 that includes the required emitters and detectors for making a SpO₂ measurement. Each of the wireless sensing devices includes an internal transceiver and battery power supply such that the sensing devices are able to transmit wireless signals 30 away from the patient 12. Each of the wireless sensing devices can also include an accelerometer that generates signals based on the posture and orientation of the patient.

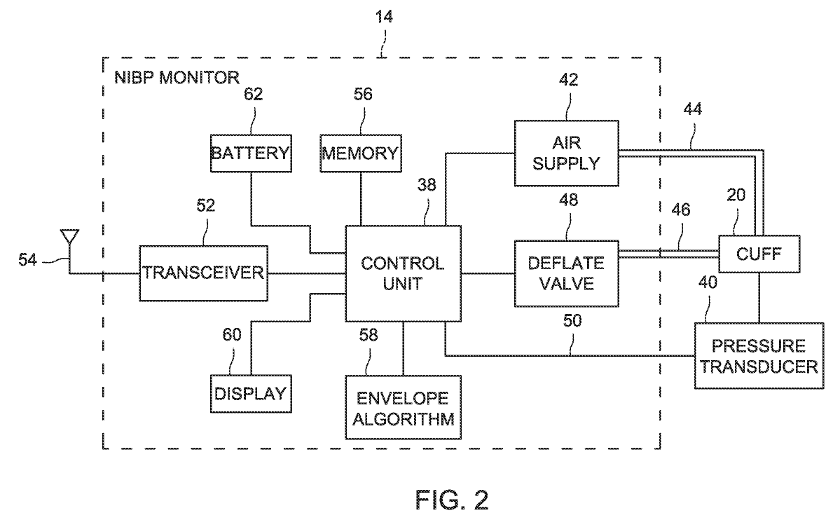

In the embodiment shown in FIG. 1, the wireless signals 30 are received at a host system, such as either the patient monitoring hub 32 or at a direct receiver for the hospital information system 34. In the embodiment shown in FIG. 2, the wireless signals from the sensing devices positioned on the patient are received at the patient monitoring hub 32 and are relayed to the hospital information system 34 through either a wireless connection, a hardwired connection or through a wireless network, such as the internet. The communication is shown in FIG. 1 by communication line 36. It is contemplated that the information from the sensors could be received directly at the hospital information system 34 or could be relayed to the hospital information system 34 through the patient monitoring hub 32.

In the embodiment shown, the blood pressure sensing device 14, the ECG sensing device 16 and the SpO₂ sensing device 18 communicate wirelessly to the patient monitoring hub 32. It is contemplated that any one of the three wireless sensing devices could be replaced by conventional sensing devices that are hardwired to either the patient monitoring hub 32 or the hospital information system 34. The use of the wireless sensing devices, as illustrated in FIG. 1, is currently contemplated as being the preferred embodiment of the present disclosure. The wireless sensing devices allow the patient 12 to move freely throughout a room or clinical environment.

FIG. 2 provides additional detail regarding the blood pressure sensing device 14. The blood pressure sensing device 14 includes a control unit 38 that includes an internal processor. The control unit 38 is used to operate all of the components of the blood pressure sensing device 14 as will be described. The blood pressure sensing device 14 includes a blood pressure cuff 20 that includes a pressure transducer 40. The control unit 38 is coupled to an air supply source 42 which provides a supply of pressurized air to the blood pressure cuff through conduit 44. A deflation conduit 46 is connected to a deflation valve 48. Through the air supply 42 and deflation valve 48, the control unit 38 is able to control the inflation and deflation of the blood pressure cuff 20 through an inflation and deflation cycle which is well known. During the inflation and deflation cycle, the pressure transducer 40 generates blood pressure data that is transmitted to the control unit along input line 50. The information from the pressure transducer is relayed by the control unit 38 to a wireless transceiver 52 where the blood pressure data can be wirelessly transmitted utilizing antenna 54. The blood pressure data can also be stored in a memory location 56 for later processing. The control unit 38 is able to retrieve one or more stored envelope algorithms 58 from a memory location. The envelope algorithms 58 are utilized by the control unit 38 to create and generate blood pressure estimations, as will be described in greater detail below. Once the control unit 38 generates the blood pressure estimate locally, the locally determined blood pressure reading can be shown on display 60. All of the components shown in the wireless blood pressure sensing device 14 are powered by an internal battery 62.

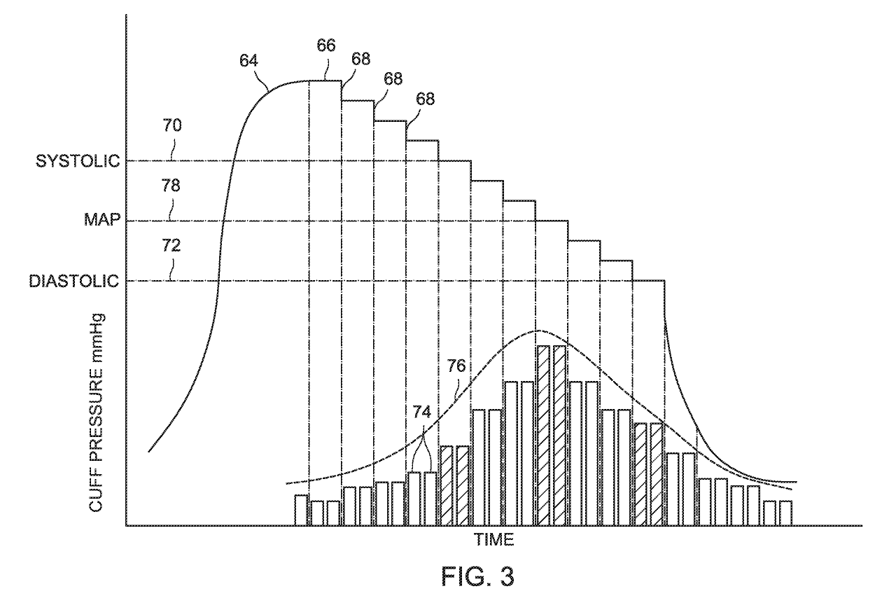

FIG. 3 illustrates a typical pressure profile 64 created by the pressure cuff. As indicated in FIG. 3, the blood pressure cuff is initially inflated to a target inflation pressure 66 that is above the systolic pressure 70 for the patient. The blood pressure cuff is deflated in a series of pressure steps 68 until the pressure within the blood pressure cuff falls below the diastolic pressure 72, after which time the blood pressure cuff is completely deflated. The pressure transducer generates a series of oscillation amplitudes 74 at each of the pressure steps 68 that are processed to create an oscillation envelope 76. Based upon the envelope 76 and the envelope algorithm, the control unit 38 is able to calculate the systolic pressure 70, diastolic pressure 72 and the mean arterial pressure (MAP) 78. The envelope algorithm, which is shown by reference numeral 58 in FIG. 2, is the algorithm needed to convert the individual oscillation amplitudes 74 into an envelope 76 that can be then used to determine the systolic, diastolic and MAP pressures for the patient. The envelope algorithm typically utilizes only the oscillation amplitudes 74 measured by the pressure transducer. However, various different multi-parameter envelope algorithms are available that provide for an enhanced blood pressure measurement by taking into account the physical characteristics of the patient as well as physiological parameters measured from the patient. As an example, if the patient is known to be suffering from preeclampsia, the algorithm can be adjusted or modified to compensate for the changes in the patient's vascular system due to this condition. Without modification, the calculated blood pressure reading would be slightly inaccurate.

In the embodiment shown in FIG. 2, the control unit 38 is able to generate the local blood pressure reading based only upon the blood pressure data received from the pressure transducer 40 and utilizing the envelope algorithm 58. However, in accordance with the present disclosure, the blood pressure data from the wireless blood pressure sensing device 14 shown in FIG. 1 can be received either at the patient monitoring hub 32 or at the hospital information system 34 and used to calculate a blood pressure reading remotely from the patient 12. The remote blood pressure reading can take into account one or more of the physiological parameters received from the patient and/or patient information retrieved from a medical records database 80. The medical records database 80 can include historic patient information, patient demographic information, past blood pressure measurements, past patient diagnoses as well as any other information that may be relevant to the determination of the blood pressure for the patient. As an example, if the patient has been previously deemed to have arterial stiffness, the diagnosis of arterial stiffness can be obtained from the medical records database 80. The ECG information received from the ECG sensing device 18 can be used to analyze the heart rythms of the patient and possibly adjust the algorithm.

As illustrated in FIG. 1, the hospital information system includes an algorithm database 82 that includes a plurality of different algorithms that can be used to determine a blood pressure reading based upon the blood pressure data obtained directly from the blood pressure sensing device 14. The selection of any one of the plurality of stored algorithms 82 can be dependent upon historic patient information retrieved from the medical records 80 or from the physiological parameters obtained from any one of the sensing devices currently attached to the patient and monitoring the physiological parameter.

During operation of the blood pressure sensing device 14 shown in FIG. 1, the blood pressure data from the blood pressure sensing device is transmitted wirelessly and received by the patient monitoring hub 32. The patient monitoring hub 32 receives the raw, unprocessed blood pressure data through the wireless transceiver 84 connected to the antenna 86. The received blood pressure data can be stored in a memory 88 for later processing by the host processor 89. The patient monitoring hub 32 includes a display 90 that can be used to present the determined blood pressure reading. The memory location 88 can store multiple blood pressure determining algorithms along with the blood pressure data obtained from the patient. The memory location 88 can also be used to store the received physiological parameters from the patient sensing devices, such as the ECG sensing device 16 and the SpO₂ sensing device 18.

As stated previously, the information received at the patient monitoring hub 32 can be relayed to the hospital information system 34 where the information is stored in a memory location 92. A host processor 94 can be used to determine the blood pressure reading at a location remote from the patient 12. Although both the patient monitoring hub 32 and the hospital information system 34 are shown including a host processor 89, 94, such that both locations can determine the blood pressure reading for the patient 12, it is contemplated that a system may only include one of the two host processors such that the remote blood pressure reading is calculated at only one location remote from the patient 12.

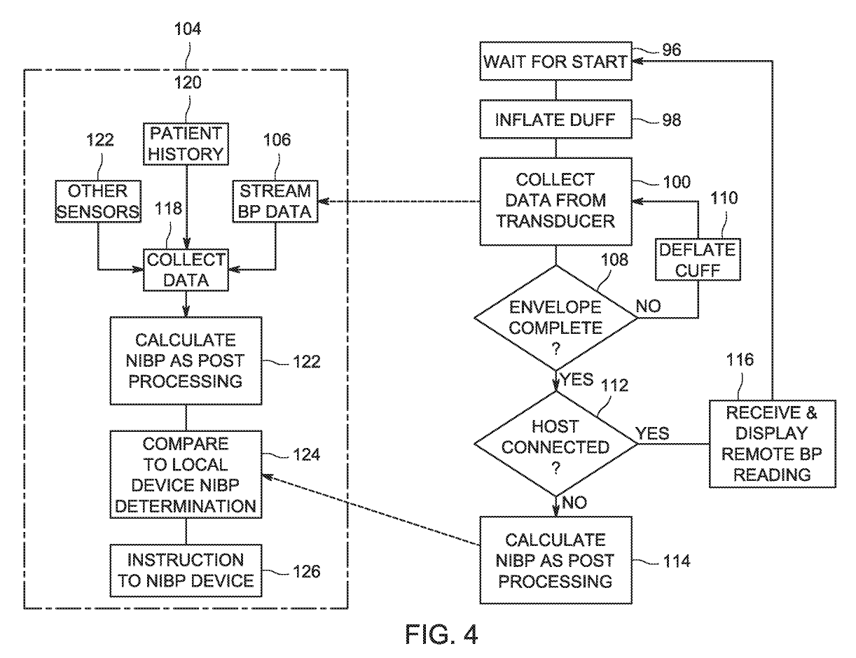

FIG. 4 illustrates one contemplated method for determining the blood pressure of a patient utilizing the system shown in FIG. 1. As shown in step 96, the blood pressure sensing device 14 initially waits for a start indicator signal, which can be received either locally at the blood pressure sensing device or as a signal received from the patient monitoring hub. Once the control unit of the blood pressure sensing device receives the start indicator, the control unit inflates the pressure cuff to a target inflation pressure. Since the blood pressure sensing device 14 is in communication with the patient hub, the control unit of the blood pressure sensing device may select the target inflation pressure based upon past blood pressure readings for the patient. In this manner, the blood pressure sensing device is able to adjust its operation based upon historic patient data to improve the overall performance of the blood pressure sensing device.

After the blood pressure cuff has been inflated, the control unit collects data from the pressure transducer in step 100. This data includes the oscillation amplitudes shown and described in FIG. 3. The blood pressure data obtained from the patient is transmitted to the host system 104. The transmission of the blood pressure data is by a wireless transmission and is received at the host system 104 as indicated in step 106. As previously described, the host system 104 could be either the patient monitoring hub or the hospital information system, or any other host system that is used to calculate the blood pressure of the patient at a location remote from the wireless blood pressure sensing device.

The control unit contained within the blood pressure sensing device proceeds to step 108 to determine whether enough data has been collected to complete the oscillation envelope, as shown by reference numeral 76 in FIG. 3. If not enough data has been collected to create the envelope, the system deflates the pressure cuff in step 110 and proceeds back to step 100 where additional data is collected. Once again, the blood pressure data is collected from the patient and is streamed to the host system 104 and stored within the memory of the wireless blood pressure sensing device.

Once enough data has been collected, the system proceeds to step 112 to determine whether the system is able to communicate with the host system 104. If the blood pressure sensing device is not able to communicate with the host system 104, the local control unit of the blood pressure sensing device calculates a local blood pressure reading in step 114. As was described in FIG. 2, the control unit 38 is able to retrieve the envelope algorithm 58 and process the blood pressure data to generate a local blood pressure reading. The local blood pressure reading can be shown on the display 60.

If the blood pressure sensing device determines that the host system 104 is connected, the system proceeds to step 116 and receives the remote blood pressure reading from the host system 104. This received remote blood pressure reading is displayed as the blood pressure reading for the patient. Since the host system 104 is able to utilize other physiological parameters from the patient as well as historic patient data, the remote blood pressure reading received from the host system will be an enhanced reading that takes into account other information to provide a more accurate and reliable blood pressure reading. However, if the host system is not in communication range, the wireless blood pressure sensing device includes enough memory and processing power to calculate a local blood pressure reading which can be read at the blood pressure sensing device.

As illustrated in FIG. 4, the host system 104 receives the streamed blood pressure data, which is collected at step 118. The blood pressure data is stored within the host system 104, such as in the memory 92 shown in FIG. 1. In addition to receiving the streamed blood pressure data, the host system retrieves the patient history at step 120 as well as other physiological parameters of the patient from various other sensors, as shown by step 122. The additional sensors could be the ECG sensing device 16 and SpO₂ sensing device 18 shown in FIG. 1. However, other patient monitoring sensors could be utilized while operating within the scope of the present disclosure. In addition, the physiological signals received from the patient could include signals from accelerometers contained in the patient sensing devices or the blood pressure sensing device. The accelerometer data can be used to determine the posture of the patient and monitor the movement of the patient.

Once the data has been collected from the blood pressure sensing device, patient sensing devices and the medical records database, the system proceeds to step 122 where the system calculates a remote blood pressure reading utilizing host processing. As described previously with reference to FIG. 1, the host system can retrieve one of multiple different stored blood pressure monitoring algorithms to enhance the blood pressure reading calculated by the host system 104. The enhanced algorithms can take into account the additional physical parameters measured from the patient, historical blood pressure readings and past patient diagnoses. It is contemplated that the remote blood pressure reading calculated in step 122 will be more accurate that the local blood pressure reading calculated by the blood pressure sensing device in step 114.

In step 124 the host processor contained within the host system 104 can receive the local blood pressure reading from the blood pressure sensing device and compare the local blood pressure reading to the remote blood pressure reading calculated by the host system. Based upon this comparison, the host system 104 can send instructions to the blood pressure sensing device, as illustrated in step 126. The instructions sent to the blood pressure sensing device can be based upon the comparison between the calculated blood pressures or could be simply based upon the physiological parameters received from the patient or information retrieved from the medical records database. The instructions sent to the wireless blood pressure sensing device could relate to the target inflation pressure, the time spent at each of the discreet pressure steps, the number of oscillation amplitudes that should be retrieved from each oscillation step as well as other information that would effect the operation of the blood pressure determination cycle carried out by the blood pressure sensing device.

As an illustrative example, if the patient parameters received from the SpO₂ monitor indicate that a large amount of noise is present, such as due to patient movement, the host system 104 may suspend operation of the blood pressure sensing device or require the blood pressure sensing device retrieve additional oscillation amplitudes at each of the pressure steps. Such modification to the operation of the blood pressure sensing device will improve performance by utilizing information that is not readily available at the blood pressure sensing device 14.

In another illustrative example, the signals from the one or more accelerometers can be used to determine the posture of the patient. Once the posture is determined, the blood pressure calculation algorithm can compensate for hydrostatic pressure changes if the blood pressure measurement device is not at the level of the patient's heart. It is known that there is an average amount of blood pressure elevation when the patient is in a sitting position compared to a lying position. Thus, the calculated pressure values can be modified to show NIBP trends that always reflect a lying position, which further enhances the accuracy of the blood pressure measurements.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

I claim:

1. A system for monitoring blood pressure in a patient, comprising:
a blood pressure sensing device configured to be worn by a patient, the blood pressure sensing device including a sensor to obtain blood pressure data from the patient and a transceiver operable to wirelessly transmit the blood pressure data;
a patient sensing device for obtaining a physiological parameter from the patient; and
a host system positioned to receive the transmitted blood pressure data and the physiological parameter, the host system including a host processor operable to calculate a remote blood pressure reading for the patient based on the received blood pressure data and the physiological parameter.

2. The system of claim 1 wherein the blood pressure sensing device further includes a control unit operable to calculate a local blood pressure reading based on the blood pressure data.

3. The system of claim 1 wherein the physiological parameter is an ECG signal.

4. The system of claim 1 wherein the physiological parameter is an SPO2 signal.

5. The system of claim 1 wherein at least one of the blood pressure sensing device and the patient sensing device includes an accelerometer that generates an orientation signal that is received by the host system.

6. The system of claim 1 wherein the physiological parameter from the patient is the orientation of the patient.

7. The system of claim 1 wherein the host system is operable to retrieve patient data from a remote medical record for the patient, wherein the host processor calculates the remote blood pressure based on the received blood pressure data, the at least one physiological parameter and the retrieved patient data.

8. The system of claim 2 wherein the host system is operable to generate control information received by the blood pressure monitoring device, wherein the control information is used to modify the calculation of the local blood pressure reading.

9. The system of claim 1 wherein the patient sensing device is configured to be worn by the patient and wirelessly transmit the physiological parameter.

10. The system of claim 1 wherein the blood pressure sensing device includes a pressure transducer operable to detect oscillometric pulses from the patient and a blood pressure cuff positionable on the patient.

11. A method of determining the blood pressure of a patient comprising the steps of:
positioning a blood pressure sensing device on the patient;
obtaining blood pressure data from the patient;
wirelessly transmitting the blood pressure data from the blood pressure sensing device;
receiving the transmitted blood pressure data at a host system;
receiving a physical parameter related to the patient at the host system;
operating a host processor to determine a remote blood pressure reading for the patient based on the received blood pressure data and the received physical parameter.

12. The method of claim 11 wherein the blood pressure sensing device further includes a control unit operable to calculate a local blood pressure reading based on the blood pressure data.

13. The method of claim 11 wherein the physiological parameter is an ECG signal.

14. The method of claim 11 wherein the physiological parameter is an SPO2 signal.

15. The method of claim 11 further comprising the step of retrieving patient data from a remote medical record for the patient, wherein the host processor calculates the remote blood pressure based on the received blood pressure data, the at least one physiological parameter and the retrieved patient data.

16. The method of claim 12 wherein the host system generates control information that is received by the blood pressure monitoring device, wherein the control information modifies the calculation of the local blood pressure reading.

17. The method of claim 11 wherein the patient sensing device is configured to be worn by the patient and wirelessly transmit the physiological parameter.

18. The method of claim 11 wherein the blood pressure sensing device includes a pressure transducer operable to detect oscillometric pulses from the patient and a blood pressure cuff positionable on the patient.

19. The method of claim 11 wherein at least one of the blood pressure sensing device and the patient sensing device includes an accelerometer that generates an orientation signal that is received by the host system.

20. The method of claim 11 wherein the physiological parameter from the patient is the orientation of the patient.
